(19) United States
(12) Patent Application Publication
(10) Pub. No.: US 2024/0197331 A1
(43) Pub. Date: Jun. 20, 2024

(54) MEDICAL DEVICE AND SYSTEM WITH ENHANCED ECHOGENICITY AND METHOD THEREOF

(71) Applicant: Coherex Medical, Inc., Salt Lake City, UT (US)

(72) Inventors: Sung K. Lee, Salt Lake City, UT (US); Tom Ditter, Mission Viejo, CA (US); Annabel Lee, Salt Lake City, UT (US)

(21) Appl. No.: 18/082,524

(22) Filed: Dec. 15, 2022

(51) Int. Cl. A61B 17/12

(52) U.S. Cl. CPC A61B 17/12122; A61B 17/12177; A61B 2017/00004; A61B 2017/1205

(57) ABSTRACT

A medical device configured to be percutaneously delivered to a heart with a catheter of a medical device delivery system. The medical device including a framework and tissue growth member. The framework includes a hub with frame segments extending radially from the hub. The tissue growth member is attached to the framework and extends radially along the framework such that the tissue growth member extends to define an outer layer portion and an inner layer portion where the framework is coupled to the inner layer portion. Further, the tissue growth member extends with multiple voids defined therein such that a filler material is configured to temporarily fill the multiple voids of the tissue growth member.

### TECHNICAL FIELD

The present invention relates generally to the occlusion of tissue openings or appendages and, more specifically, to devices, systems and methods for enhancing imaging of implants during or subsequent to a procedure intended to occlude tissue openings or appendages including, for example, left atrial appendages.

### BACKGROUND

The upper chambers of the heart, the atria, have appendages attached to each of them. For example, the left atrial appendage is a feature of all human hearts. The physiologic function of such appendages is not completely understood, but they do act as a filling reservoir during the normal pumping of the heart. The appendages typically protrude from the atria and cover an external portion of the atria. Atrial appendages differ substantially from one to another. For example, one atrial appendage may be configured as a tapered protrusion while another atrial appendage may be configured as a re-entrant, sock-like hole. The inner surface of an appendage is conventionally trabeculated with cords of muscular cardiac tissue traversing its surface with one or multiple lobes.

The atrial appendages appear to be inert while blood is being pumped through them during normal heart function. In other words, the appendages don't appear to have a noticeable effect on blood pumped through them during normal heart function. However, in cases of atrial fibrillation, when the atria go into arrhythmia, blood may pool and thrombose inside of the appendages. Among other things, this can pose a stroke risk when it occurs in the left appendage since the thrombus may be pumped out of the heart and into the cranial circulation once normal sinus rhythm is restored following arrhythmia events.

Historically, appendages have sometimes been modified surgically to reduce the risk imposed by atrial fibrillation. In recent years devices which may be delivered percutaneously into the left atrial appendage have been introduced. The basic function of these devices is to exclude the volume within the appendage with an implant which then allows blood within the appendage to safely thrombose and then to be gradually incorporated into cardiac tissue. This process, coupled with the growth of endothelium over the face of the device, can leave a smooth, endothelialized surface where the appendage is located. In comparison to surgical procedures, devices implanted percutaneously are a less invasive means for addressing the problems associated with the left atrial appendage.

During implantation of the device the physician typically uses a sound transmitting instrument, such as, transesophageal echocardiography (TEE) to monitor the location of the device during the procedure. However, the materials generally used to form the device, which promote endothelialization, such as, for example, polymeric materials, typically include properties or characteristics of hydrophobicity, cavities and porosity that may trap air within the one or more layers of the polymeric materials. The trapped air prevents sound energy to transmit through the device and emit a complete structural image of the device to the physician, thus making it difficult for the physician to determine and monitor the location of the device during implantation. This misdiagnosis of location may result in positioning the device in a less than optimal position and/or orientation than what is intended and may increase the risk of effusion.

### BRIEF SUMMARY OF THE INVENTION

The present invention is directed to a medical device system configured to occlude an opening in a heart. The medical device system includes a delivery system including a handle and a catheter. The medical device system also includes an implant removably coupled to the catheter. The implant includes a framework and a tissue growth member. The framework includes a hub with frame segments extending radially from the hub. The tissue growth member is attached to the framework. The tissue growth member extends radially along the framework such that the tissue growth member extends to define an outer layer portion and an inner layer portion, the framework being coupled to the inner layer portion. The tissue growth member extends with multiple voids defined therein such that the tissue growth member includes a filler material configured to temporarily fill the multiple voids of the tissue growth member.

In another embodiment, the filler material is configured to fill the multiple voids defined along an outer surface of the outer layer portion. In a further embodiment, the filler material is configured to fill the multiple voids defined in portions below the outer surface of the outer layer portion.

In another embodiment, the tissue growth member includes etched holes defined in the tissue growth member, the etched holes extending through each of the outer layer portion and the inner layer portion and the etched holes of the tissue growth member receive a second filler material.

In another embodiment, the filler material includes a bio-dissolvable material. In a further embodiment, the bio-dissolvable material includes a bio-absorbable material.

In another embodiment, the outer layer portion includes multiple ePTFE layers. In a further embodiment, the tissue growth member includes a polyurethane material. In another further embodiment, the inner layer portion includes a laminated polymeric material, the laminated polymeric material configured to attach to the framework of the implant.

In accordance with another embodiment of the present invention, a medical device configured to be percutaneously delivered to a heart with a catheter of a medical device delivery system is provided. The medical device includes a framework and tissue growth member. The framework includes a hub with frame segments extending radially from the hub. The tissue growth member is attached to the framework, the tissue growth member extends radially along the framework such that the tissue growth member extends to define an outer surface and an inner surface. The framework is coupled to the inner surface of the tissue growth member. The tissue growth member extends with multiple voids defined therein, the tissue growth member including a filler material configured to temporarily fill the multiple voids of the tissue growth member.

In one embodiment, the filler material is configured to fill the multiple voids defined along the outer surface of the tissue growth member. In a further embodiment, the filler material is configured to fill the multiple voids defined in portions below the outer surface of the tissue growth member.

In another embodiment, the tissue growth member includes formed holes defined in the tissue growth member. In another embodiment, the formed holes of the tissue growth member are formed as etched holes extending through each of the outer surface and the inner surface of the tissue growth member. In another embodiment, the formed holes of the tissue growth member are formed as drilled holes extending through each of the outer surface and the inner surface of the tissue growth member. The formed holes extending through each of the outer surface and the inner surface are configured to be filled with a second filler material. In another embodiment, the filler material comprises a bio-dissolvable material.

In accordance with another embodiment of the present invention, a method for occluding an opening in a heart is provided. The method includes the step of: providing a medical device system extending to define a delivery system and an implant, the delivery system including a handle and a catheter, the catheter being removably coupled to the implant, the implant extending to define a framework, the framework including a hub with frame segments extending radially from the hub, the framework being attached to a tissue growth member, the tissue growth member extending radially along the framework to define an outer layer portion and an inner layer portion, the framework coupled to the inner layer portion, the tissue growth member extending to define multiple voids therein, the tissue growth member including a filler material configured to temporarily fill the multiple voids of the tissue growth member; and positioning the implant within a left atrial appendage of the heart.

In another embodiment, the providing step includes providing the tissue growth member with etched portions extending through each of the outer layer portion and the inner layer portion of the tissue growth member. In still another embodiment, the providing step includes providing the etched portions with a second filler material. In another embodiment, the providing step includes providing the tissue growth member with at least one of a polyurethane material and an ePTFE material.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 a perspective view of a medical device system, depicting an implant coupled to a delivery system and an anchor portion of the implant in an engageable position, according to one embodiment of the present invention;

FIG. 1A is an enlarged partial cross-sectional view of the medical device system taken along section line 1A-1A of FIG. 1, depicting a framework of the implant and tines of the anchor portion of the implant in the engageable position, according to another embodiment of the present invention;

FIG. 2 is a perspective view of the medical device system, depicting the anchor portion of the implant in a retracted position, according to another embodiment of the present invention;

FIG. 2A is an enlarged partial cross-sectional view taken along section line 2A-2A of FIG. 2, depicting an anchor hub of the implant in a proximal position to move the anchor portion to the retracted position, according to another embodiment of the present invention;

FIG. 3 is a perspective view of the medical device system, the implant being at least partially constricted within a sheath of the medical device system, according to another embodiment of the present invention;

FIG. 4 is an enlarged view taken from region 4 of FIG. 2A, depicting detail of a tissue growth member of the implant of the medical device system, according to another embodiment of the present invention;

FIG. 4A is a simplified enlarged view of a portion of the tissue growth member in a pre-formed state, depicting a primary member including apertures therein, according to another embodiment of the present invention;

FIG. 4B is a simplified enlarged view of the portion of the tissue growth member, depicting a filler material being deposited onto the primary member to fill the apertures of the primary material, according to another embodiment of the present invention;

FIG. 4C is a simplified enlarged view of additional portions of the tissue growth member, depicting the filler material being deposited onto a first layer of a secondary member of the tissue growth member, according to another embodiment of the present invention;

FIG. 4D is a simplified enlarged view of additional portions of the tissue growth member, depicting the filler material being deposited onto a second layer of the secondary member and the first layer coupled to the primary member, according to another embodiment of the present invention;

FIG. 4E is a simplified enlarged view of additional portions of the tissue growth member, depicting the filler material being deposited onto a third layer of the secondary member and the second layer being coupled to the first layer, according to another embodiment of the present invention;

FIG. 4F is a simplified enlarged view of the tissue growth member, depicting a framework positioned adjacent the tissue growth member and the third layer being coupled to the second layer, according to another embodiment of the present invention;

FIG. 4G is a simplified enlarged view of the tissue growth member, depicting the framework being coupled to the primary member, according to another embodiment of the present invention;

FIG. 5 is a simplified enlarged view of another embodiment of a tissue growth member coupled to the framework of the implant, according to the present invention;

FIG. 5A is a simplified enlarged view of a portion of the tissue growth member of FIG. 5, depicting a primary member including apertures therein, according to another embodiment of the present invention;

FIG. 5B is a simplified enlarged view of the portion of the tissue growth member, depicting a filler material deposited onto and filling the apertures of the primary member, according to another embodiment of the present invention;

FIG. 5C is a simplified enlarged view of an additional portion of the tissue growth member, depicting a secondary member positioned adjacent the primary member, according to another embodiment of the present invention;

FIG. 5D is a simplified enlarged view of the tissue growth member positioned adjacent the framework, according to another embodiment of the present invention;

FIG. 6 is a simplified enlarged view of another embodiment of a tissue growth member coupled to the framework of the implant, according to the present invention;

FIG. 6A is a simplified enlarged view of a portion of the tissue growth member, depicting a primary member including apertures therein, according to another embodiment of the present invention;

FIG. 6B is a simplified enlarged view of the primary member of the tissue growth member, depicting a filler material deposited onto and filling the apertures of the primary member, according to another embodiment of the present invention;

FIG. 6C is a simplified enlarged view of additional portions of the tissue growth member, depicting a secondary member positioned adjacent the primary member, according to another embodiment of the present invention;

FIG. 6D is a simplified enlarged view of the additional portions of the tissue growth member, depicting the secondary member coupled to the primary member, according to another embodiment of the present invention;

FIG. 6E is a simplified enlarged view of portions of the tissue growth member, depicting the portions of the tissue growth member being etched to form cavities in the tissue growth member, according to another embodiment of the present invention;

FIG. 6F is a simplified enlarged view of the portions of tissue growth member, depicting the tissue growth member receiving an embedded material being deposited thereon to fill the cavities defined in the tissue growth member, according to another embodiment of the present invention;

FIG. 6G is a simplified enlarged view of the tissue growth member, depicting the tissue growth member including the embedded material deposited in the cavities of the tissue growth member, according to another embodiment of the present invention;

FIG. 6H is a simplified enlarged view of the tissue growth member, depicting the tissue growth member being positioned adjacent the framework, according to another embodiment of the present invention;

FIG. 7 is a simplified enlarged view of another embodiment of a tissue growth member coupled to a framework, depicting the tissue growth member having a filler material formed with a portion of the tissue growth member and a textile embedded therein, according to the present invention;

FIG. 8 is a simplified enlarged view of another embodiment of a tissue growth member coupled to a framework, depicting the tissue growth member having a filler material formed therewith and the tissue growth member having multiple layers formed over a primary layer, according to the present invention;

FIG. 9 is a simplified enlarged view of another embodiment of a tissue growth member coupled to a framework, depicting the tissue growth member having a filler material formed therewith, according to the present invention; and

FIG. 10 is a simplified enlarged view of another embodiment of a tissue growth member coupled to a framework, depicting the tissue growth member having a filler material formed therewith, according to the present invention.

### DETAILED DESCRIPTION OF THE INVENTION

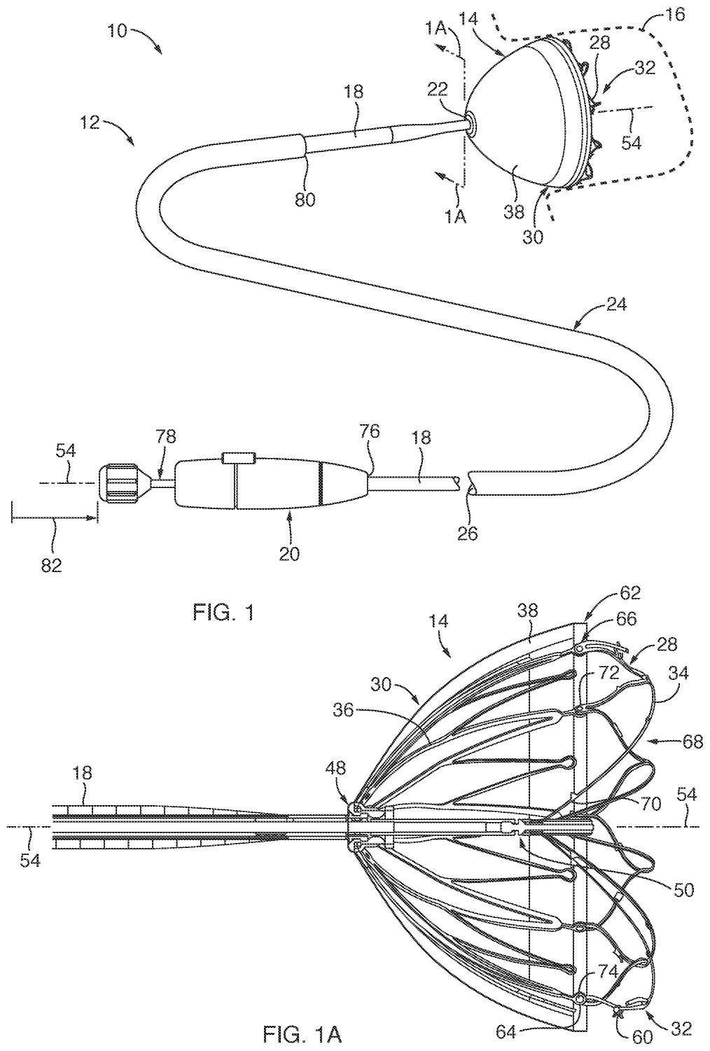

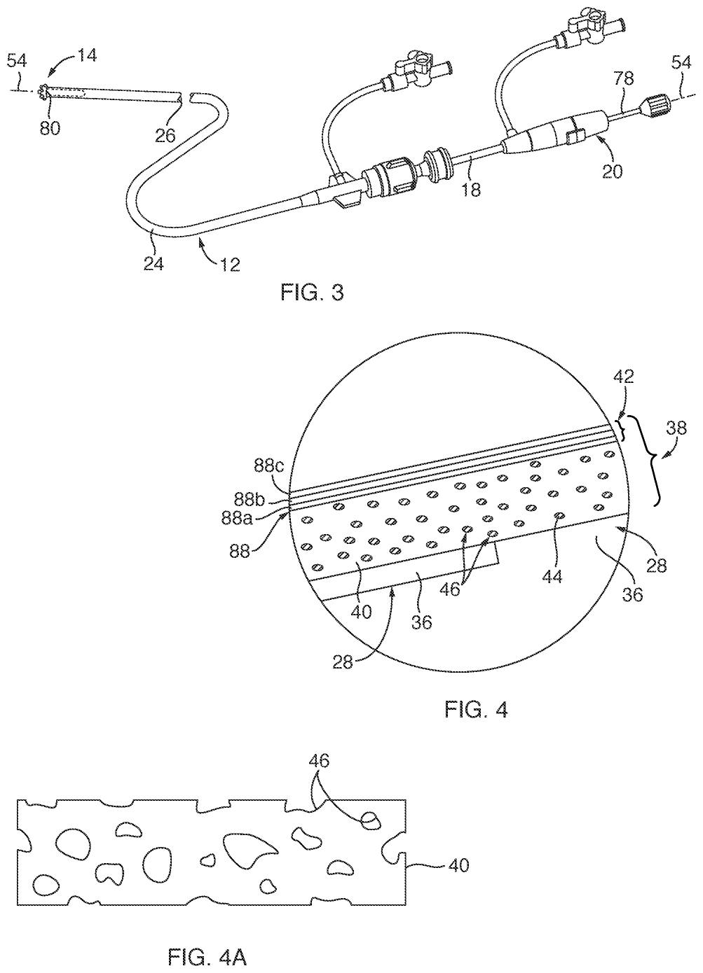

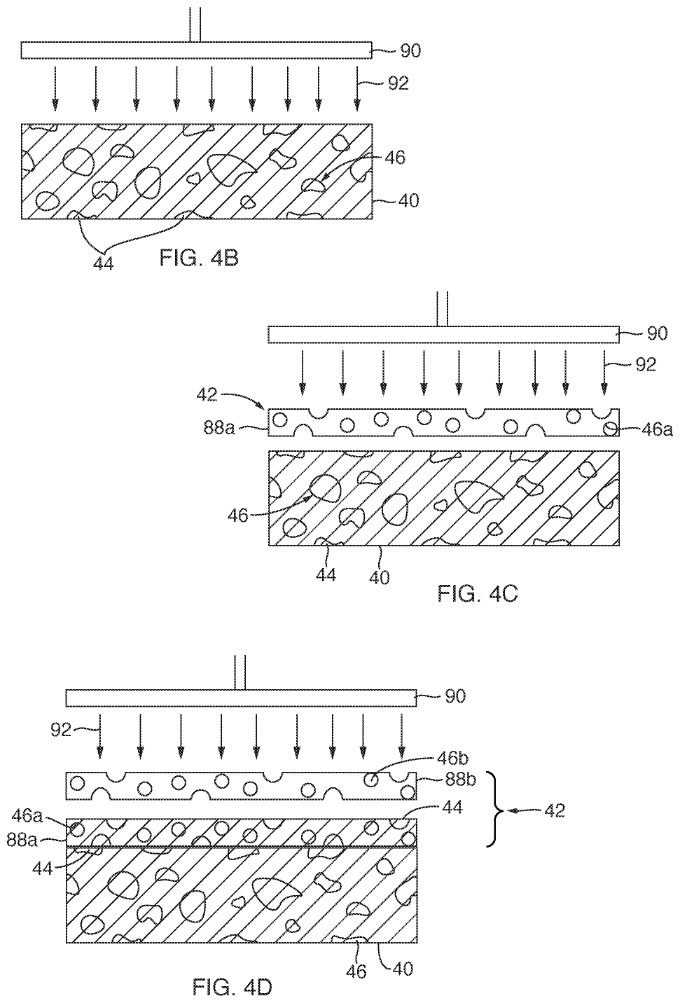

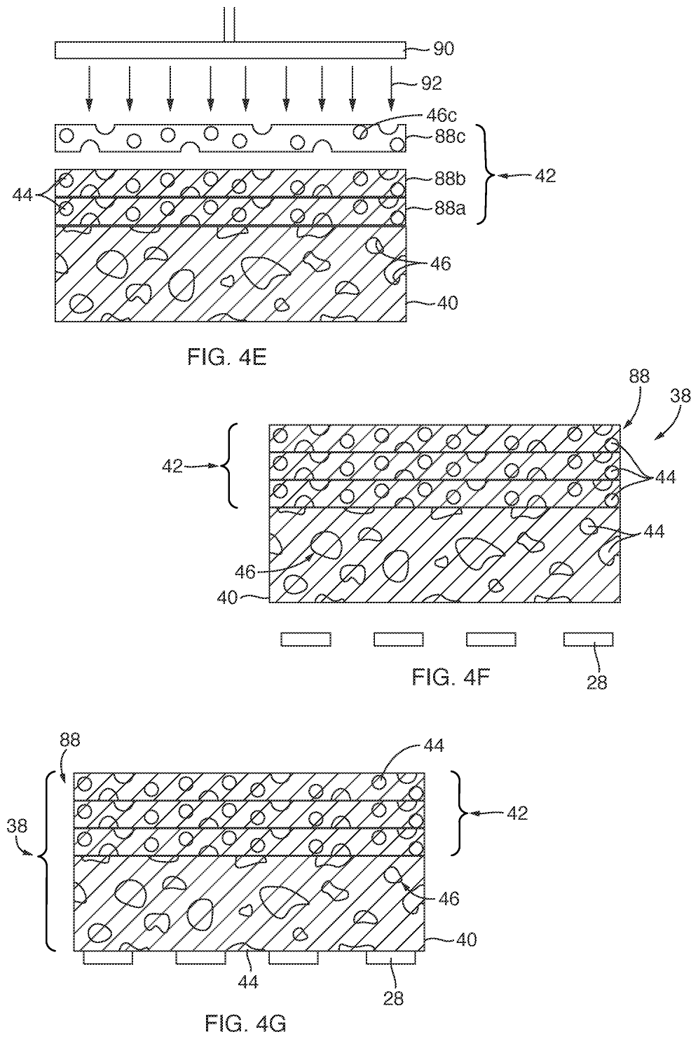

Referring to FIGS. 1, 1A and 4, a medical device system 10 including a delivery system 12 and an implant 14 such that the implant 14 may be removably coupled to the delivery system 12 is provided. The medical device system 10 may be employed in interventional procedures for percutaneously closing and modifying an opening or cavity such as, for example, a left atrial appendage 16 within a heart (not shown). The delivery system 12 may include a pusher catheter 18 and a handle 20, the pusher catheter 18 being coupled to the implant 14 adjacent a distal end 22 of the pusher catheter 18. Also, the delivery system 12 may include a sheath 24 sized and configured to facilitate advancing the implant 14 through a lumen 26 of the sheath 24 with the pusher catheter 18 (see also FIG. 3). In addition, the implant 14 may extend to define a framework 28 extending along an occluder portion 30 and an anchor portion 32. The anchor portion 32 may extend with an anchor frame 34 and the occluder portion 30 may include an occluder frame 36 with a tissue growth member 38 attached to the occluder frame 36.

The tissue growth member 38 may include multiple materials or layers of materials, such as a primary member 40 and a secondary member 42. In one embodiment, the primary member 40 and/or the secondary member 42 may include multiple apertures 46 defined therein. Such apertures 46 may also be referenced as recesses or cavities that may be natural characteristics of the materials that may also result in voids or air pockets between the various layers of the tissue growth member 38 or throughout the primary member 40. Such layers of the tissue growth member 38 may include a filler material or coating that may fill such apertures of the tissue growth member 38. Such apertures being filed or covered may enhance the viewability of imaging employed with a sound type imaging device, such as ultrasound or transesophageal echocardiography (“TEE”). The filler material implemented with the tissue growth member 38 may enhance echogenicity or the imaging of the implant 14 and, thus, the ability of the physician to appropriately position and anchor the implant 14 within the human anatomy, such as the left atrial appendage 16. In addition, the filler material 44 may be a bio-dissolvable material or biodegradable material configured to fill (or partially fill) the apertures 46 defined throughout the tissue growth member 38. In this manner, the filler material 44 many eliminate many of the voids in the tissue growth member 38 so as to make the tissue growth member more solid so that the implant may be better viewed with the sound type imaging and, thus, the implant 14 may be better positioned in the human anatomy. Further, the filler material 44 or coating of all (or most) voids and cavities may lower the surface energy so that the tissue growth member 38 exhibits structural characteristics of being hydrophilic or super-hydrophilic so that the filled voids or cavities enhance the visibility under sound energy or sound type imaging. In one embodiment, the tissue growth member 38 may be exposed or receive UV light thereto for a predetermined period of time so that the filler material 44 and/or tissue growth member 38 may oxidize and become more hydrophilic. Upon implanting the device, over a predetermined period of time, the filler material on the implant 14 may be absorbed in the human body or degrade naturally so that the original surface of the tissue growth member 38 may be exposed so that endothelialization may naturally occur over the implant 14 in, for example, the left atrial appendage.

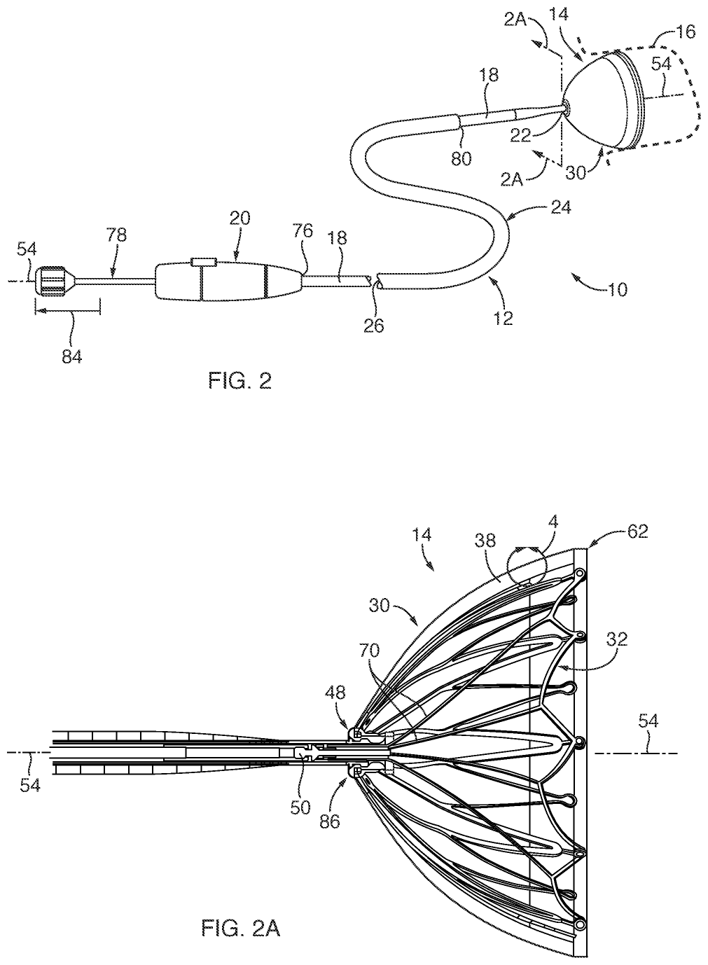

As depicted in FIGS. 1 and 1A, the implant 14 may include a primary hub 48 and a secondary hub 50 such that the framework 28 may extend between the primary hub 48 and the secondary hub 50. The primary hub 48 may define a bore and an axis 54 that each may extend through the primary hub 48 such that the axis 54 may extend axially relative to the bore and structure of the primary hub 48. Such axis 54 may also extend axially along a length of the delivery system 12 and the components thereof. Further, the secondary hub 50 may be moveable along the axis 54 and through the primary hub 48 so as to move the anchor portion 32 of the framework 28 between a retracted position or constricted position (FIG. 2A) and a deployed position (FIG. 1A).

As previously set forth, the framework 28 may extend with the occluder portion 30 to define the occluder frame 36 and the framework 28 may extend with the anchor portion 32 to define the anchor frame 34. The anchor frame 34 may extend with the anchor portion 32 and define anchor tines 60 extending therefrom. The occluder frame 36 may extend with the occluder portion 30 with the tissue growth member 38 attached to the occluder frame 36. The tissue growth member 38 may be in the form of an occlusive member, but may also be in the form of a filter member, a mesh member, a membrane or any other structure, or combinations thereof, sized and configured to promote tissue in-growth. Further, the tissue growth member 38 may be formed from one or more polymeric materials, such as ePTFE and/or a polyurethane foam. Even further, the tissue growth member 38 may extend with multiple layers with varying thicknesses and sizes.

Further, the occluder frame 36 may be coupled to the primary hub 48 such that the occluder frame 36 may extend radially outward from the primary hub 48 and may extend distally to an occluder frame 36 distal end 62. Adjacent to the distal end 62 of the occluder frame 36, the occluder frame 36 may include multiple occluder frame eyelets 64 defined in the occluder frame 36. The anchor frame 34 may extend between a first end 66 and a second end 68, the first end 66 coupled to the occluder frame 36 and the second end 68 coupled to the secondary hub 50. The anchor frame 34 may extend with multiple anchor frame segments 70, interconnected to each other, extending between the first and second ends 66, 68 of the anchor frame 34. Adjacent to the first end 66 of the anchor frame 34, the anchor frame 34 may include multiple anchor frame eyelets 72 along multiple ones of the anchor frame segments 70 of the anchor frame 34. At the secondary hub 50, multiple ones of the anchor frame segments 70 or anchor frame extensions may be coupled to the secondary hub 50. Each of the occluder frame eyelets 64 may be coupled to a corresponding one of the anchor frame eyelets 72 with a hinge component 74. The hinge component 74 may extend through the occluder frame eyelet 64 and the anchor frame eyelet 72 so as to facilitate the anchor frame 34 to pivot about the hinge component 74 so as to pivot or rotate relative to the occluder frame 36. With this arrangement, the anchor frame 34 may be pivotably coupled (or hingeably coupled) to the occluder frame 36 so that the anchor frame 34 may move between the retracted position (FIG. 2A) and the deployed position (FIG. 1A). The retracted position of the anchor frame 34 may also be an anchor constrained position or pivoted position. As such, the pivoting between the retracted and deployed positions of the anchor frame 34 may assist a physician in adjusting the position of the implant 14 subsequent to the anchor portion 32 being secured to tissue in, for example, the left atrial appendage 16.

With reference to FIGS. 1-3, as previously set forth, the implant 14 may be delivered through the vasculature with the delivery system 12. The delivery system 12 may include the pusher catheter 18 and the handle 20, the handle 20 integrated with a proximal portion 76 of the pusher catheter 18. The handle 20 may include various functional components, such as an anchor actuator 78, to manipulate the anchor frame 34 between the deployed position (FIG. 1A) and the retracted position (FIG. 2A). The delivery system 12 may include and be employed with a sheath 24 for delivering the implant 14 through the vasculature and to the left atrial appendage 16 in the heart. The sheath 24 may be positioned within the vasculature using known interventional techniques with a sheath distal end 80 to be positioned adjacent the left atrial appendage 16 of the heart. Upon the implant 14 being advanced through the lumen 26 of the sheath 24 to the sheath distal end 80 (the implant 14 being in the constricted position partially shown in dashed lines adjacent the sheath distal end 80 (see FIG. 3)), the implant 14 may at least partially be deployed from the sheath 24. That is, the sheath 24 may then be manually moved proximally (and/or the pusher catheter 18 advanced distally) so that the occluder portion 30 of the implant 14 may be deployed from the sheath distal end 80 (see FIG. 2). Such occluder portion 30 may immediately self-expand as the occluder portion 30 is exposed from the sheath distal end 80. At this stage, the implant 14 may be in a partially deployed state, after which, the implant 14 may be moved to a fully deployed state by deploying the anchor portion 32 (see FIG. 1). For example, upon the occluder portion 30 initially being deployed, the anchor portion 32 may be in the retracted position with the anchor actuator 78 of the handle 20 in the proximal position (as depicted in FIG. 2). Once a physician determines that the occluder portion 30 is in an appropriate and desired position adjacent the left atrial appendage 16, the anchor portion 32 may be pivoted from the retracted position to the deployed position by moving the anchor actuator 78 to the distal position, as shown by arrow 82 (see FIG. 1). Once the anchor portion 32 is moved to the deployed position, the anchor tines 60 (FIG. 1A) of the anchor portion 32 may engage tissue to secure the implant 14 in the left atrial appendage 16. If the physician determines that the implant 14 is not in an optimal secured position in the left atrial appendage 16, the anchor portion 32 may be pivoted back to the retracted position by moving the anchor actuator 78 from the distal position to the proximal position, as shown by arrow 84 (see FIG. 2). As such, the anchor actuator 78 may be manually moved proximally and distally to move the anchor portion 32 between the retracted and deployed positions such that the anchor portion 32 pivots between the deployed and retracted positions. In this manner, the anchor portion 32 of the implant 14 may be secured and disengaged from tissue in the left atrial appendage 16 as needed by the physician until the physician obtains an optimal position or is satisfied with its position prior to releasing the delivery system 12 from the implant 14. A similar delivery system and implant is disclosed in commonly assigned U.S. patent application Ser. No. 15/438,650, filed on Feb. 21, 2017, now issued as U.S. Pat. No. 10,631,969 entitled MEDICAL DEVICE FOR MODIFICATION OF LEFT ATRIAL APPENDAGE AND RELATED SYSTEMS AND METHODS, the disclosure of which is incorporated by reference herein in its entirety.

Now with reference to FIGS. 2A and 4, as previously set forth, the tissue growth member 38 may be formed from one or more polymeric materials, such as ePTFE and/or a polyurethane foam and extend with multiple layers of varying thicknesses, sizes, and materials. The tissue growth member 38 may include the primary member 40 attached to the occluder frame 36 and may extend along the length of the occluder portion 30 from a proximal end 86 to the distal end 62 of the occluder portion 30. The primary member 40 may be formed from one or more polymeric materials, such as ePTFE, polyurethane foam, polyester based materials, PTFE materials, and/or polyurethane materials formed through an electrospinning process or any other suitable process for forming a polymeric material. Further, the primary member 40 may be made from a material which may define apertures 46 throughout the primary member 40. The apertures 46 may have a high internodal distance or a low internodal distance where the internodal distance is the relative size of the apertures 46 defined throughout the primary member 40. The tissue growth member 38 may also include a secondary member 42, the secondary member 42 being attached or coupled, through adhesion or stitching, to the primary member 40 and extending along the primary member 40 from a proximal end 86 to a distal end 62 of the occluder portion 30. The secondary member 42 may be made of one or more layers 88, such as a first layer 88a, a second layer 88b, and a third layer 88c. The layers 88 may be formed from one or more polymeric materials, such as ePTFE and/or polyurethane foam, such that each layer 88 may consist of a separate material. The secondary member 42 may also include apertures 46 defined throughout the layers 88 of the secondary member 42, further, the apertures 46 defined in the secondary member 42 may consist of different internodal distances such that, for example, the apertures 46 defined in the first layer 88a may have a high internodal distance while the apertures 46 of the second and third layers 88b, 88c may have a low internodal distance. The apertures 46 of the secondary member 42 and primary member 40 may create air pockets throughout the primary and secondary members 40, 42 which may promote tissue growth. The apertures 46 defined throughout the secondary member 42 may have a different internodal distance, such as a lower internodal distance, than the apertures 46 defined throughout the primary member 40. The tissue growth member 38 may include the filler material 44 configured to fill in the apertures 46 defined throughout the tissue growth member 38, such that the tissue growth member 38 may appear to be a more solid structure via sound imaging techniques, as previously set forth.

In one embodiment, the filler material 44 may fill in the apertures 46 of the primary member 40, while any apertures that may be defined in the secondary member 42 may be not receive the filler material 44. In another embodiment, the filler material 44 may fill in the apertures 46 defined throughout the primary member 40 and any one of the first layer 88a, the second layer 88b, and the third layer 88c of the secondary member 42. As previously set forth, the filler material 44 may be formed from a bio-dissolvable or biodegradable material configured to dissolve or degrade over time with contact to bodily fluids such that the apertures of the tissue growth member 38 may become exposed. Such exposed apertures may then assist in promoting an endothelial layer and tissue growth over the implant 14.

With reference to FIGS. 4 and 4A-4G, one embodiment of a method or steps to form the tissue growth member 38 with the filler material 44 is provided. As previously set forth, the tissue growth member 38 may include the primary member 40 and secondary member 42, where, the secondary member 42 may include one or more layers 88. As depicted in FIG. 4A, the primary member 40 may include the apertures 46 therein, the apertures 46 may be air pockets, defined throughout the primary member 40. Further, the primary member 40 may be formed from a polymeric material, such as polyurethane foam. As depicted in FIG. 4B, the filler material 44 may be applied with the application device 90 by either coating, soaking, and/or spraying the filler material 44 to the primary member 40, as depicted by arrows 92. Upon applying the filler material 44 to the primary member 40, the filler material 44 may fill in the apertures 46 so as to minimize voids and air pockets associated with the primary member 40.

As depicted in 4C, the secondary member 42 may include a first layer 88a with apertures 46a or recesses therein. The first layer 88a may be a polymeric material, such as ePTFE, or any other suitable polymeric material. The apertures 46a of the first layer 88a may be smaller than the apertures 46 of the primary member 40, but such sizing may be dependent on the polymeric material of the first layer 88a and the primary member 40. The filler material 44 may be applied by the applicator device 90 to fill in the apertures 46a of the first layer 88a of the secondary member 42, as shown by arrows 92, similar to that previously depicted for the primary member 40.

As depicted in FIG. 4D, the first layer 88a of the secondary member 42 may then be coupled to the primary member 40 with an adhesive such as glue or other means which may adhere the first layer 88a to the primary member 40. Further, the secondary member 42 may include a second layer 88b with apertures 46b formed therein, similar to the first layer 88a. The second layer 88b may be formed from a polymeric material similar to the first layer 88a, such as ePTFE. In another embodiment, the second layer 88b may be formed from a polymeric material different to the first layer 88a of the secondary member 42. In one embodiment, the second layer 88b may be formed of a similar type material, but with different sized apertures 46b than the apertures 46a of the first layer 88a. In another embodiment, the apertures 46b defined in the second layer 88b may be similar in size or similar size range to the apertures 46a defined in the first layer 88a when the first and second layers 88a, 88b are formed from a similar type of polymeric material, such as ePTFE. In another embodiment, the apertures 46b defined in the second layer 88b may be dissimilar from the apertures 46a defined in the first layer 88a if the first and second layers 88a, 88b are formed from a different polymeric material. As depicted, the filler material 44 may be applied by the applicator device 90, as depicted by arrows 92, to fill in the apertures 46b of the second layer 88b of the secondary member 42 so that voids or air pockets defined by the apertures 46b defined in the second layer 88b may be minimized.

As depicted in FIG. 4E, the second layer 88b of the secondary member 42 may be coupled to the first layer 88a such that the primary member 40, first layer 88a, and second layer 88b are coupled together. Further, the secondary member 42 may include a third layer 88c. The third layer 88c may include apertures 46c formed therein, similar to the first and second layers 88a, 88b of the secondary member 42. Further, similar to the first and second layers 88a, 88b, the third layer 88c may be formed from a polymeric material. Further, the filler material 44 may be applied by the applicator device 90 to the third layer 88c of the secondary member 42, in a similar manner as applied to the first layer 88a and the second layer 88b. As depicted in FIG. 4F, the third layer 88c of the secondary member 42 may be coupled to the second layer 88b of the secondary member 42 to form the various layers of the tissue growth member 38, similar to that depicted in FIG. 4. As depicted in FIGS. 4F and 4G, such tissue growth member 38 may be coupled to the framework 28 with an adhesive and/or via attachment processes, such as by hooking or tying, or the like. As depicted, the underside of the primary member 40 of the tissue growth member 38 may be directly coupled to one side of the framework 28 so that the primary member 40 may extend along the framework 28. In another embodiment, each of the first layer 88a, second layer 88b, and third layer 88c may be adhesively coupled together prior to receiving the application of the filler material 40 so that the secondary member 42 (comprising each of the first, second and third layers 88a, 88b, 88c) may then be coupled to the primary member 40. In another embodiment, the secondary member 42 may include additional or fewer layers, each of which may be polymeric layers, such as ePTFE.

As previously set forth, in one embodiment, the filler material 44 applied to the primary member 40 as well as the secondary member 42 may be a bio-dissolvable or biodegradable material. In another embodiment, the filler material 44 may only be applied to the primary member 40. In another embodiment, a different type of filler material 44 may be applied to the secondary member 42 than that which is applied to the primary member 40 such that the filler material 44 may dissolve at different rates. For example, the filler material 44 applied to the secondary member 42 may dissolve or absorb into the anatomy rapidly in comparison to the filler material 44 applied to the primary member 40. As such, effective imaging of the implant 14 may be employed during the procedure of delivering the implant 14, positioning the implant 14, anchoring the implant 14 and/or releasing the implant 14 in the anatomy, as previously set forth herein, while the filler material 44 along the secondary member 42 may dissolve or absorb into the anatomy so that the apertures of the secondary member 42 may become exposed sooner than the apertures of the primary member 40. Upon the filler material 44 of the secondary member 42 being absorbed into the anatomy, the apertures along the outer surface of the secondary member 42 (or outer surface of the tissue growth member 38, which may be the portion of the tissue growth member 38 directly contacting tissue) may become exposed so that the endothelialization layer may be more effectively initiated along such outer surface of the secondary member 42 at an early stage of the implant 14 being released in the anatomy, such as in the left atrial appendage. Further, due to the filler material 46 of the primary member 40 being configured to dissolve at a slower rate, the imaging viewability of the implant 14 may continue to be effective for a period of time subsequent to the implant 14 being released in the anatomy.

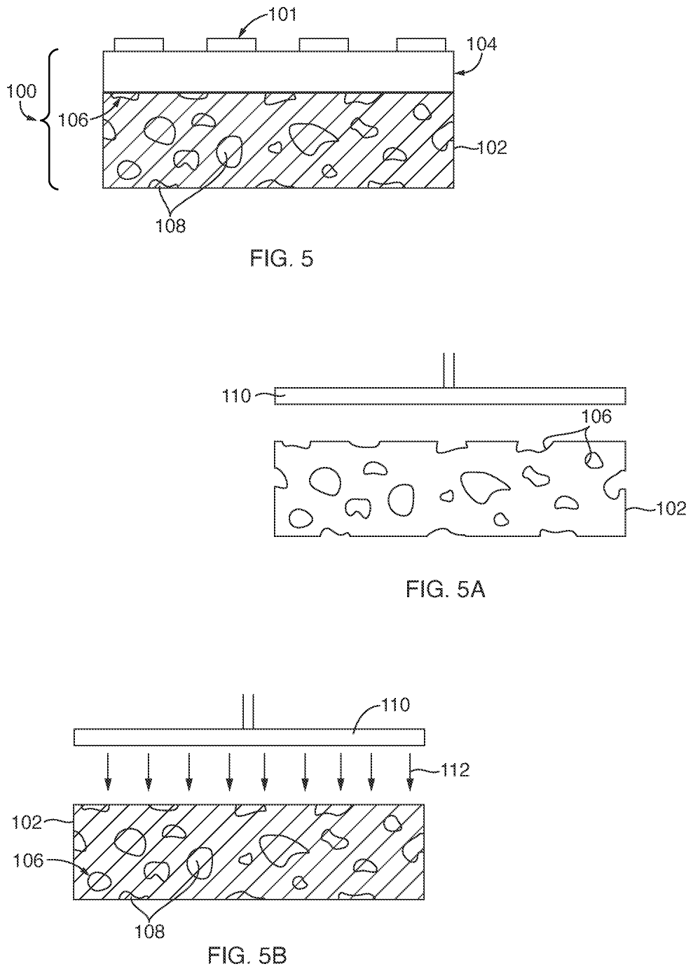

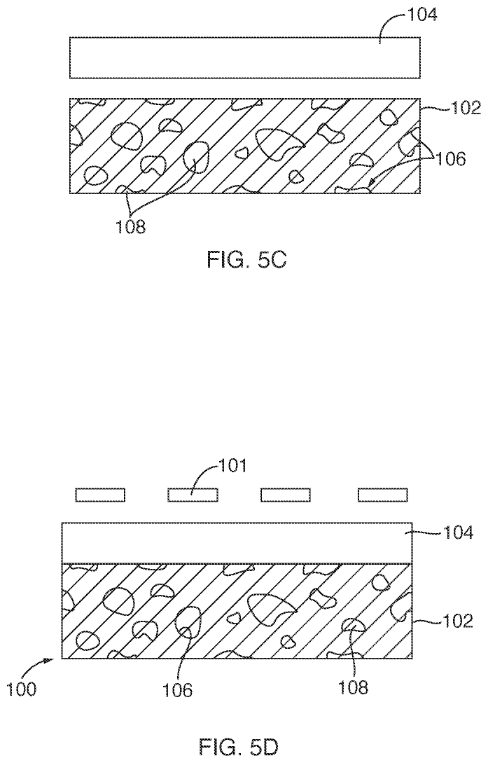

Now with reference to FIGS. 5, and 5A-5D, another embodiment of a tissue growth member 100 attached to a framework 101, similar to the framework 28 of the implant 14 depicted in FIG. 1, and a method for forming such tissue growth member 100 is provided. In this embodiment, the tissue growth member 100 may include a primary member 102 coupled to a secondary member 104. Further, the primary member 102 may be a polymeric material with multiple apertures 106 defined therein, the apertures 106 being fillable with a filler material 108. As in the previous embodiment, the filler material 108 may be a biodegradable or bio-dissolvable material that may be absorbed into the body upon the implant being implanted therein, such as within a left atrial appendage. Further, the secondary member 104 may also be formed from a polymeric material. In this embodiment, the secondary member may be coupled directly to the framework 101. The primary member 102 may be formed from a polymeric material with, for example, an electrospinning process or the like. The polymeric material employed with the electrospinning process may include polyester based materials, a polyurethane material or a polytetrafluoroethylene material or any other suitable polymeric material that is a biocompatible polymer. Such electrospinning process may provide randomly located apertures 106 or cavities formed in the primary member 102 that may leave voids and air pockets in the primary member 102. As such, similar to previous embodiments, the primary member 102 may be treated so as to receive the filler material 108 to fill the apertures and form a denser structure. The secondary layer 104 may also be formed from a polymeric material, such as a polyurethane material or the like.

Now with reference to FIGS. 5A-5D, another embodiment for forming the tissue growth member 100 with the filler material is provided. For example, in one embodiment, the primary member 102 with its apertures 106 formed therein may be positioned adjacent an application device 110, as depicted in FIG. 5A. As such, the application device 110 may apply the filler material 108 to the primary member 102, as depicted by arrows 112, such that the filler material may fill all or some of the apertures 106 of the primary member 102. The secondary member 104 may then be positioned adjacent the primary member 102, as depicted in FIG. 5C. The secondary member 104 may be a sheet material formed of a polymeric material, such as a polyurethane material. The secondary member 104 may be sized to correspond with the sizing of the primary member 102 and may be attached to the primary member with, for example, an adhesive, as depicted in FIG. 5D, to form the tissue growth member 100. Further, as depicted in FIG. 5D, the tissue growth member 100 may be positioned adjacent to the framework 101 so that the tissue growth member may be attached to the framework 101 (see FIG. 5) with an adhesive and/or stitching, for example. In one embodiment, the secondary member 104 may be laminated so that the secondary member 104 may readily bond to the framework 101. In this manner, the tissue growth member 100 may be formed with the filler material 108 and employed with an implant, similar to the implant 14 set forth in FIG. 1, so that the implant may be readily viewable with sound type imaging techniques. As in previous embodiments, the filler material 108 of the tissue growth member 100 of the implant may be a bio-dissolvable or biodegradable material that may dissolve after being implanted into the body so that the apertures 106 defined in the primary layer 102 may be exposed and act to promote an endothelialization layer and eventually promote tissue growth to the tissue growth member 100.

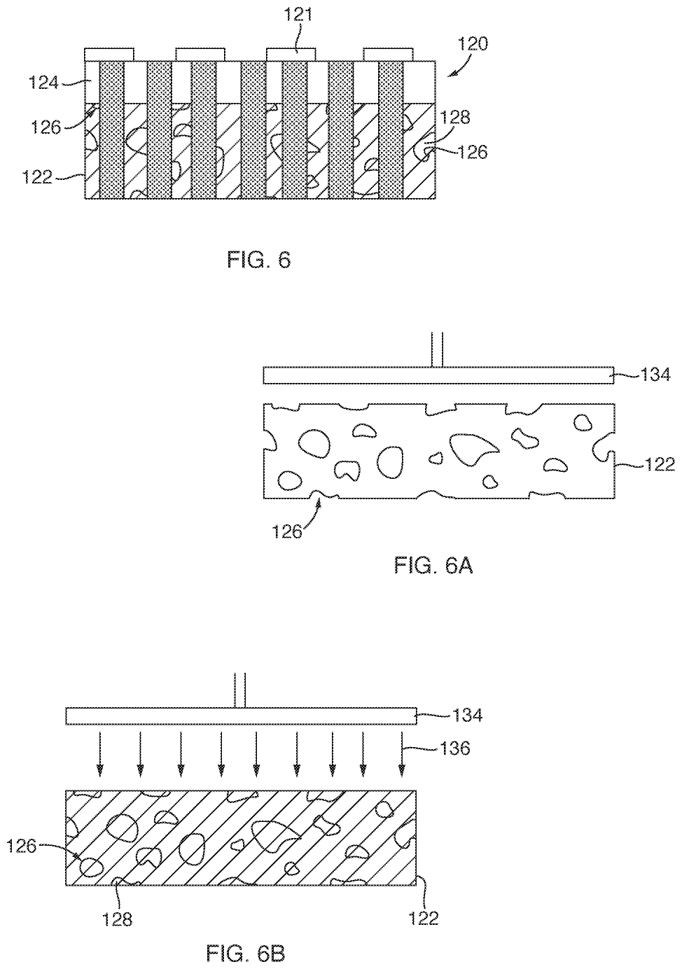

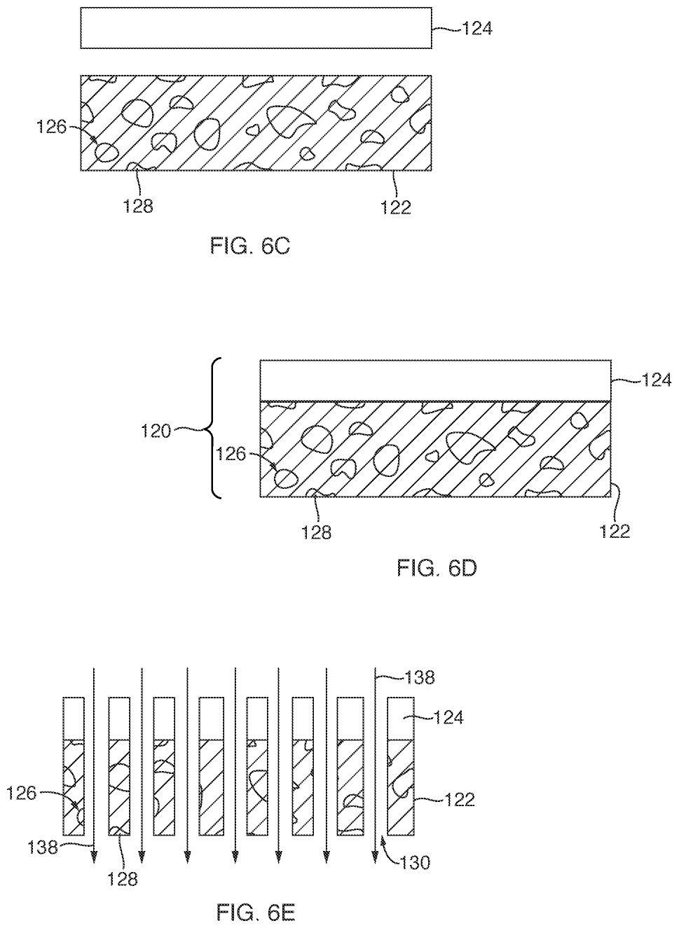

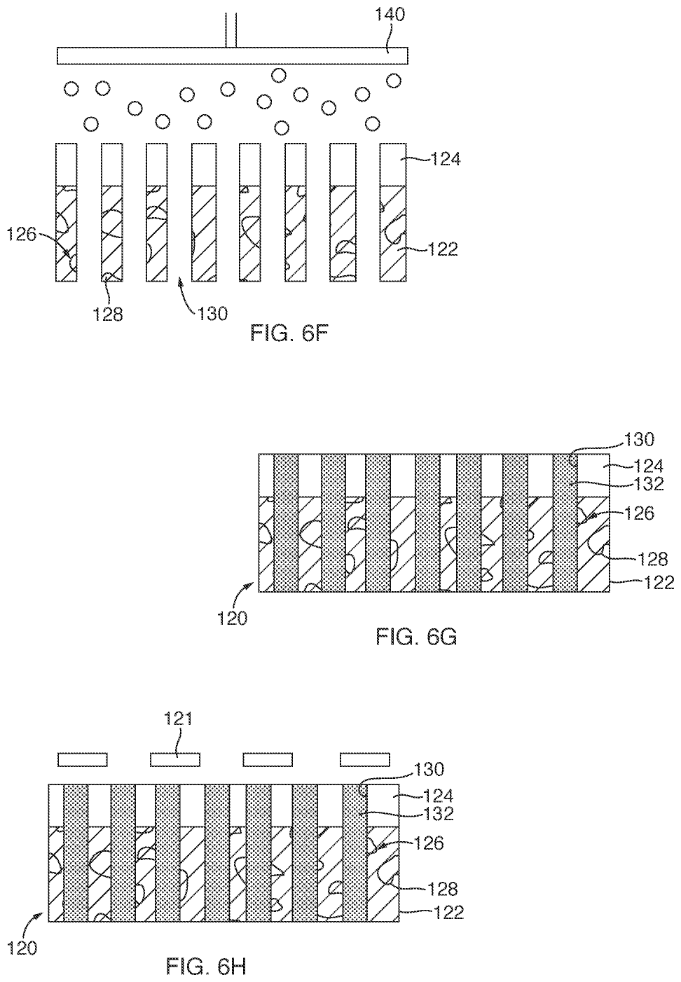

With reference to FIGS. 6, and 6A-6H, another embodiment of a tissue growth member 120 for attaching to the framework 121 of an implant, such as the framework 28 of implant 14 depicted in FIG. 1, is provided. For example, as depicted in FIG. 6, the tissue growth member 120 may include a primary member 122 and a secondary member 124, the secondary member 124 being coupled to the primary member 122. Further, the primary member 122 includes apertures 126 therein, which may be filled with a fillable material 128, similar to other embodiments set forth herein. Further, in this embodiment, tissue growth member 120 may include holes 130 formed through the tissue growth member 120 and an imbedded material 132. Such formed holes 130 may be filled with the imbedded material 132. The imbedded material 132 may be designed and configured to be a sound amplifying structure, such as acoustic impedance matching pillars or the like, or any type of biocompatible material that may assist in amplifying sound to enhance sound type imaging techniques. In one embodiment, the primary member 122 may be an ePTFE material with recesses or cavities generally having a first internodal distance, the recesses and cavities being the before described apertures 126. The secondary member 124 may also be an ePTFE material with recesses or cavities generally having structural characteristics of a second internodal distance, the second internodal distance being smaller than the first internodal distance. In another embodiment, the primary member 122 may be a polyester based material, a polyurethane material or a polytetrafluoroethylene material or combinations thereof that may be formed with an electrospinning process, similar to the previous embodiment.

Now with reference to FIGS. 6A-6H, one embodiment of a method for forming the tissue growth member 120 is provided. As depicted in FIGS. 6A and 6B, similar to previous embodiments, the primary member 122 may be positioned adjacent an applicator device 134. The apertures 126 of the primary member 122 may be filled with the fillable material 128 applied by the applicator device 134, as depicted by arrows 136. Upon the primary member 122 receiving the fillable material 128, the secondary member 124 may be positioned adjacent the primary member 122 and then coupled to the primary member 122, as depicted in FIGS. 6C and 6D. As depicted in FIG. 6E, the primary member 122 and the secondary member 124 may then undergo an etching process, as depicted by arrows 138, to form the holes 130 through the primary member 122 and/or the secondary member 124. In another embodiment, the primary member 122 and the secondary member 124 may undergo drilling or the like to form the holes 130, or any other suitable process for forming holes, as known to one of ordinary skill in the art. As depicted in FIG. 6F, the primary member 122 and secondary member 124 may then receive the imbedded material 132 with a deposition device 140, for example. The imbedded material 132 may then fill the etched holes 130, as depicted in FIG. 6G, to thereby form the tissue growth member 120. The tissue growth member 120 may then be positioned adjacent the framework 28, as depicted in FIG. 6H, so that the tissue growth member 120 may be attached to the framework 28, as shown in FIG. 6. In this manner, the filler material and the imbedded material of the tissue growth member facilitates enhancing the echogenicity of the tissue growth member and, thus, the implant during implantation of the implant

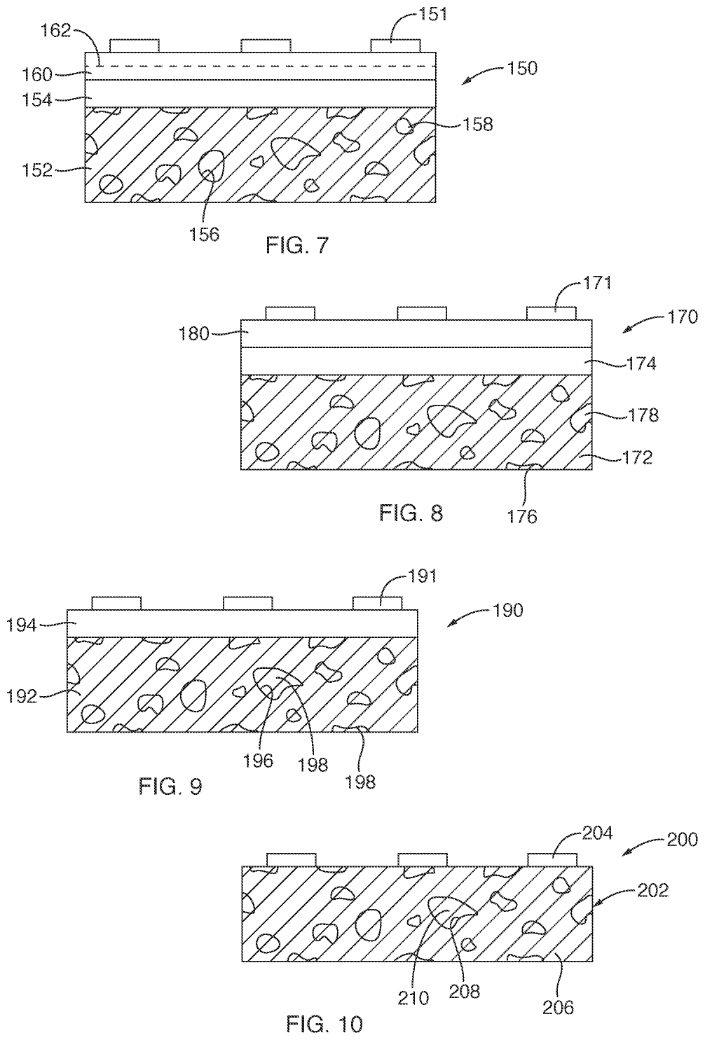

With reference to FIG. 7, another embodiment of a tissue growth member 150 that may be employed with a framework 151, similar to the framework 28 and tissue growth member 38 of the implant 14 depicted in FIGS. 1 and 1A. In this embodiment, the tissue growth member 150 may include a primary member 152 and a secondary member 154, the primary member 152 having apertures 156 formed therein. As in previous embodiments, the apertures 156 may be filled with the filler material 158 and the secondary member 154 may be coupled to the primary member 152. Further, similar to the previous embodiment, the primary member 152 and the secondary member 154 may a polymeric layer, such as an ePTFE material. In this embodiment, the tissue growth member 150 may include a third member 160 that may include an embedded textile 162 therein. The textile 162 may be embedded to the third layer 160 through a lamination process by heating the third member 160 and the textile 162 so that the textile 162 bonds thereto, as known to one of ordinary skill in the art. The third member 160 may also be coupled to the framework 151 of the implant (not shown), as well as to the secondary member 154. As in previous embodiments, the filler material 158 may be a bio-dissolvable or biodegradable material that may dissolve after being implanted into the body so that the apertures 156 defined in the primary member 152 may be exposed and act to promote an endothelialization layer and eventually promote tissue growth to the tissue growth member 150.

With reference to FIG. 8, another embodiment of a tissue growth member 170 that may be coupled to a framework 171 of an implant (not shown), similar to the framework 28 and tissue growth member 38 of the implant 14 described relative to FIGS. 1 and 1A. In this embodiment, the tissue growth member 170 may include a primary member 172 and a secondary member 174 such that the primary member 172 may include apertures 176 formed therein, the apertures 176 being filled with a filler material 178. The primary and second members 172, 174 may be formed from an ePTFE material, similar to the previous embodiment. The tissue growth member 170 may also include a third member 180, but without the textile reinforcement structure. The third member 180 may be coupled to the framework 171 and may act as an underlayment for bonding to and protecting the primary and secondary members 172, 174 of the tissue growth member 170. Similar to previous embodiments, the filler material 178 of the tissue growth member 170 may be a bio-dissolvable or biodegradable material that may dissolve after being implanted into the body so that the apertures 176 defined in the primary member 172 may be exposed and act to promote an endothelialization layer and eventually promote tissue growth to the tissue growth member 170.

With reference to FIG. 9, another embodiment of a tissue growth member 190 that may be coupled to a framework 191 of an implant (not shown), similar to the framework 28 and tissue growth member 38 of the implant 14 described in FIGS. 1 and 1A. This embodiment of the tissue growth member 190 may include a primary member 192 and a secondary member 194 such that the primary member 192 may include apertures 196 formed therein, the apertures 196 being filled with a filler material 198, similar to previous embodiments. In this embodiment, the secondary member 194 may be coupled to the primary member 192 as well as coupled to the framework 191. Further, in this embodiment, the primary member 192 may be an ePTFE material and the secondary member 194 may be an underlayment sheet of, for example, a polyurethane sheet or the like similar to the third member of the previous embodiment. As such, the secondary member 194 may act as an underlayment for bonding to and protecting the primary member 192 that may readily be coupled to the framework 191. As in previous embodiments, the filler material 198 may be a bio-dissolvable or biodegradable material that may dissolve from the tissue growth member 190 after being implanted into the body so that the apertures 196 defined in the primary member 192 (or outer layer) of the tissue growth member 190 may be exposed and act to promote an endothelialization layer and eventually promote tissue growth to the tissue growth member 190.

With reference to FIG. 10, in another embodiment, a tissue growth member 202 may be coupled to a framework 204 of an implant 200, similar to the framework 28 and tissue growth member 38 of the implant 14 described in FIGS. 1 and 1A. However, in this embodiment, the tissue growth member 202 may only include a primary member 206. In one embodiment, the tissue growth member 202 may not be a layered tissue growth member. In another embodiment, the primary member 206 of the tissue growth member 202 may be a molded polymeric member. In still another embodiment, the primary member 206 of the tissue growth member 202 may be a molded polymeric member with layers added thereto, similar to that depicted in previous embodiments, such as the embodiments depicted in FIGS. 4, 5, 6, 7, 8 and 9. The primary member 206 may include apertures 208 formed therein. Similar to previous embodiments, the apertures 208 defined in the primary member 206 may be filled with a filler material 210. Further, in this embodiment, the primary member 206 may be coupled or attached to the framework 204 of the implant 200. The primary member 206 may be any suitable polymeric material. As in previous embodiments, the filler material 210 may be bio-dissolvable or biodegradable material that may dissolve after being implanted into the body so that the apertures 208 defined in the primary member 206 may become exposed over time and act to promote an endothelialization layer and eventually promote tissue growth to the tissue growth member 202.

While the invention may be susceptible to various modifications and alternative forms, specific embodiments have been shown by way of example in the drawings and have been described in detail herein. Further, the structural features of any one embodiment disclosed herein may be combined or replaced by any one of the structural features of another embodiment set forth herein. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A medical device system configured to occlude an opening in a heart, the medical device system comprising:
a delivery system including a handle and a catheter; and
an implant removably coupled to the catheter, the implant including:
a framework including a hub with frame segments extending radially from the hub; and
a tissue growth member attached to the framework, the tissue growth member extending radially along the framework such that the tissue growth member extends to define an outer layer portion and an inner layer portion, the framework coupled to the inner layer portion, the tissue growth member extending with multiple voids defined therein, the tissue growth member including a filler material configured to temporarily fill the multiple voids of the tissue growth member.

2. The medical device system of claim 1, wherein the filler material is configured to fill the multiple voids defined along an outer surface of the outer layer portion.

3. The medical device system of claim 2, wherein the filler material is configured to fill the multiple voids defined in portions below the outer surface of the outer layer portion.

4. The medical device system of claim 1, wherein the tissue growth member comprises etched holes defined in the tissue growth member, the etched holes extending through each of the outer layer portion and the inner layer portion, the etched holes of the tissue growth member receiving a second filler material.

5. The medical device system of claim 1, wherein the filler material comprises a bio-dissolvable material.

6. The medical device system of claim 5, wherein the bio-dissolvable material comprises a bio-absorbable material.

7. The medical device system of claim 1, wherein the outer layer portion comprises multiple ePTFE layers.

8. The medical device system of claim 1, wherein the tissue growth member comprises a polyurethane material.

9. The medical device system of claim 1, wherein the inner layer portion comprises a laminated polymeric material, the laminated polymeric material configured to attach to the framework of the implant.

10. A medical device configured to be percutaneously delivered to a heart with a catheter of a medical device delivery system, the medical device comprising:
a framework including a hub with frame segments extending radially from the hub; and
a tissue growth member attached to the framework, the tissue growth member extending radially along the framework such that the tissue growth member extends to define an outer surface and an inner surface, the framework coupled to the inner surface of the tissue growth member, the tissue growth member extending with multiple voids defined therein, the tissue growth member including a filler material configured to temporarily fill the multiple voids of the tissue growth member.

11. The medical device of claim 10, wherein the filler material is configured to fill the multiple voids defined along the outer surface of the tissue growth member.

12. The medical device of claim 11, wherein the filler material is configured to fill the multiple voids defined in portions below the outer surface of the tissue growth member.

13. The medical device of claim 10, wherein the tissue growth member comprises formed holes defined in the tissue growth member, the formed holes extending through each of the outer surface and the inner surface of the tissue growth member, the formed holes of the tissue growth member configured to receive a second filler material.

14. The medical device system of claim 10, wherein the filler material comprises a bio-dissolvable material.

15. A method for occluding an opening in a heart, the method comprising:
providing a medical device system extending to define a delivery system and an implant, the delivery system including a handle and a catheter, the catheter being removably coupled to the implant, the implant extending to define a framework, the framework including a hub with frame segments extending radially from the hub, the framework being attached to a tissue growth member, the tissue growth member extending radially along the framework to define an outer layer portion and an inner layer portion, the framework coupled to the inner layer portion, the tissue growth member extending to define multiple voids therein, the tissue growth member including a filler material configured to temporarily fill the multiple voids of the tissue growth member; and
positioning the implant within a left atrial appendage of the heart.

16. The method according to claim 15, wherein the providing comprises providing the tissue growth member with etched portions extending through each of the outer layer portion and the inner layer portion of the tissue growth member.

17. The method according to claim 16, wherein the providing comprises providing the etched portions with a second filler material.

18. The method according to claim 15, wherein the providing comprises providing the tissue growth member with at least one of a polyurethane material and an ePTFE material.
